(19) United States
(12) Patent Application Publication
(10) Pub. No.: US 2005/0107635 A1
(43) Pub. Date: May 19, 2005

(54) Metoprolol manufacturing process

(76) Inventors: Janakraj Karamchand Mehra, Mumbai (IN); Ajit Choubey, Sejavta (IN); Bimal Kumar Srivastava, Sejavta (IN); Rajendra Kumar Porwal, Sejavta (IN); Prashant Gautam, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/807,221

(22) Filed: Mar. 23, 2004

(30) Foreign Application Priority Data
Nov. 14, 2003 (IN) 1185/MUM/2003

(51) Int. Cl. C07C 217/32

(52) U.S. Cl. 564/349

(57) ABSTRACT

Metoprolol manufacturing process with optimized reaction temperatures and reactant molar ratios, to avoid the manufacture of excessive epoxide intermediates, thus avoiding the need for purification of epoxide intermediates, thus achieving higher yields and higher-purity product than that seen in the prior art teachings.

### RELATED APPLICATIONS

This application claims priority from (1) “An Improved Industrial Process For Manufacture Of Metoprolol Base And Salts Thereof,” India provisional patent application Serial No. 1185/MUM/2003, filed 14 Nov. 2003; and from (2) “An Improved Industrial Process For Manufacture Of Metoprolol Base And Salts Thereof,” India utility patent application Serial No. 1185/MUM/2003, filed 7 Jan. 2004, the contents of which are incorporated by reference here.

### GOVERNMENT INTEREST

None.

The following specification describes the nature of the invention.

### TECHNICAL FIELD OF THE INVENTION

This invention relates to an improved industrial process for manufacture of compounds useful as β-blocker, antihypertensive compounds, more particularly an improved industrial process for manufacture of Metoprolol base and salts thereof.

### BACKGROUND

Metoprolol and its salts such as its tartrate and succinate salts are well established drugs having anti-hypertensive activity. These compounds acts as β-blockers. The patients suffering from hypertension needs, however, to be on treatment by these drugs for their whole lifetime. This kind of therapy necessitates that the drugs are of high purity with minimal impurity levels, so that the adverse side effect caused by impurities is minimum. There is also a demand to produce these kinds of drugs at cheaper prices.

Ample literature is available for producing metoprolol base and its salts, due to the significance of these compounds as anti hypertensive agents. Spanish patent No. ES 2,011,584 (the national equivalent is U.S. Pat. No. 5,082,969) describes a process for manufacturing metoprolol, where 4-(2-methoxyethyl)phenol and epichlorohydrin are reacted in aqueous alkaline conditions at 0°-25° C. temperature for 15-20 hours. The organic phase consisting of epoxide is separated, washed with water and used as such for reaction with large excess of isopropylamine in aqueous media like water at 0°-30° C. temperature.

Polish Patent PL 158,497 describes a process wherein 4-(2-methoxyethyl)phenol and epichlorohydrin are reacted at 20°-80° C. temperature for 3 hours under aqueous alkaline conditions. The epoxide so formed is reacted with large excess of isopropyl amine (medium as well as reactant) to yield metoprolol base.

Perhaps the nearest prior art to the present invention is the form of U.S. Pat. No. 6,252,113, which discloses reaction of 4-(2-methoxyethyl)phenol and epichlorohydrin in aqueous alkaline conditions at 50°-70° C. temperature for 1 hour. The resulting epoxide is distilled under high vacuum to improve quality. Pure epoxide then is treated with isopropylamine in solvent such as isopropyl alcohol.

As has been seen, a lot of knowledge in the present field of invention is available; however, there remain some problems, which problems defined the scope for our further investigations. It can be seen that almost all prior art have the same reactants which are the same. Reactions which are carried out at lower temperatures (below ambient room-temperature) leads to a relatively slow rate of reaction; in contrast, more impurity levels occur when the reaction is carried out at higher temperature range, so that while the rate of reaction is increased, the resulting product requires purification by distillation under a high vacuum.

In the processes where purification of the epoxide is avoided, the resultant products are formed with higher impurities. The processes involving excess use of isopropyl amine leads to increased costs. The products formed with higher impurity levels necessitate extra purifications, which increases the cost of manufacturing the product.

Therefore, it is of importance to develop a process for manufacture of metoprolol base, which process is economical, eco-friendly and yielding high quality with higher yields, which also avoids the expense-increasing purification operations like high vacuum distillation.

The patient suffering from hypertension needs to be on treatment by these drugs for their whole lifetime. This kind of therapy therefore necessitates that the drugs be of extremely high purity with very low impurity levels, so that the adverse side effects attributable to impurities are minimal. There is also a demand to produce these drugs at cheaper prices.

### OBJECTIVES

An objective of the present invention is therefore to develop a process to manufacture Metoprolol base and salts thereof, in high yields, with higher purity, and with better operator-friendly operations, at cheaper prices.

### SUMMARY

This invention relates to an improved industrial process for manufacturing Metoprolol base and salts thereof.

### DETAILED DESCRIPTION

The present invention has made it possible to produce metoprolol base and its salts in higher yields, and with high purity, and avoiding processes like high-vacuum distillation, thus enabling cheaper manufacturing costs.

The present invention involves optimization of reaction temperatures and the molar ratio of reactants in order to achieve higher-purity and higher yields, by avoiding the excessive manufacture of epoxide intermediates seen in the prior art teachings, and thus avoiding the need for purification of these epoxide intermediates.

The present invention process involves three steps. The first step is for preparation of epoxide by reacting 4-(2-methoxyethyl)phenol with epichlorohydrin in an aqueous media containing inorganic base such as sodium hydroxide at 40-45° C. temperature, rather than the lower temperature range taught by U.S. Pat. No. 5,082,969, nor the higher temperature range taught by U.S. Pat. No. 6,252,113.

As with the prior art teachings, at the end of the reaction, the aqueous and organic phases are separated out. In contrast to the prior art teachings, however, in our process the organic phase is washed one or more times by water, and we have found that the pH of the washing water for at least the last washing must be in the range of pH 7 to 8; this pH range is necessary to achieve high purity of the epoxide.

The resultant epoxide is used in the second step for preparation of metoprolol base. The epoxide is treated with isoproplyamine in aqueous media to obtain Metoprolol base of high purity in high yields. The last step is converting metoprolol base into the succinate and tartrate salts, by reacting the metoprolol base with an acid (such as succinic acid or tartaric acid) in solvent media (such as acetone) by any conventional method. This last step—reacting in an organic solvent such as acetone, rather than in an aqueous media—contrasts with the teachings of the art, which discourage using organic solvents (see e.g., U.S. Pat. No. 5,082,969 at column 2, lines 18-28 (“aqueous medium . . . present[s] notable advantages with respect to other processes which require organic solvents”); U.S. Pat. No. 6,252,113 at col. 1, lines 37-44 (“The difference from the prior art is that the new method uses no other solvents than water for the reaction of A and B. From an environmental as well as an occupational hazard point of view it is a great advantage to be able to replace a hazardous organic solvent with a non-noxious solvent such as water.”)).

By following the present invention as described below, it has been made possible to produce metoprolol base and its salts in higher yields with high purity and avoiding processes like high vacuum distillation, at cheaper costs.

The present invention involves optimization of reaction temperatures, molar ratio of reactants in order to achieve higher purity and yields by avoiding purification of epoxide intermediates.

The general process involves three steps:
Step—1 : reacting 2-(methoxyethyl)phenol with epichlorohydrin to form epoxide. The organic phase is washed thrice by water with pH in the range of 7 to 8.
Step—2: reacting epoxide with isopropyl amine to form Metoprolol base.
Step—3: reacting metoprolol base to form a Metoprolol salt.

The process of the present invention is illustrated by the following example.

### EXAMPLE 1

Step 1 : Epoxide Formation

Into a reaction vessel are measured 4-(2-methoxyethyl)phenol and epichlorohydrin, in an appropriate molar ratio. The molar ratio of 4-(2-methoxyethyl)phenol to epichlorohydrin is in the range of 1:0.92 to 1:2.0. The more-preferred ratio is 1:1.1 to 1:1.4, and the most-preferred ratio is 1:1.31, to leave a product of maximum purity.

The 4-(2-methoxyethyl)phenol and epichlorohydrin are mixed in aqueous media like water. Our currently-preferred ratio of 4-(2-methoxyethyl)phenol:water is 1:1.6 volumes. To this mixture is added an inorganic base such as sodium hydroxide. Our currently-preferred concentration of sodium hydroxide in water is 25% weight/volume; our currently-preferred molar ratio of sodium hydroxide to 4-(2-methoxyethyl)phenol is 1.14:0.95, more preferably is 1.024:1, and most preferably ratio is 1.136:1.

This mixture is reacted at temperature range of 40° to 45° C., for 3 to 5 hours time.

At the end of the reaction, the aqueous and organic phases are separated out. The organic phase is washed thrice by water. The pH of washing must be in the range of 7 to 8. This pH range is necessary to achieve high purity of the epoxide.

Traces of water are removed from organic phase by azeotropic distillation under vacuum below 55° C. temperature. By keeping the residue below 55° C., under vacuum for 3 to 5 hours, the purity of sample can achieve a range of 97 to 99%. The yield of the epoxide, so obtained is in the range of 93 to 95% of the theoretical stoicheometric maximum, and a purity of 97 to 99%. The epoxide with this purity is used for making metoprolol base.

Step 2 : Metoprolol Base

Our currently-preferred temperature during addition of epoxide is 10° to 25° C. Our currently-preferred molar ratio of epoxide:isopropyl amine is 1:5.25±0.25; our most-preferred ratio is 1:5.25±0.05.

After addition of epoxide, the epoxide and isopropyl amine mixture is reacted in aqueous media (e.g., water) at a temperature of not greater than 30° C. Our currently-preferred ratio of water to epoxide is 2:1 volume:weight.

At the maximum temperature of 30° C, the reaction completes in about 3 hours time. In order to achieve high purity it is necessary to maintain a temperature range of about 5° C to not more than about 25° C. The reaction works below 5° C., albeit is sluggish Formation of impurity at RRT 0.35 (as measured by gas chromatography) is minimized by operating at not more than about 30° C. We have observed that in cases where the temperature rises above the range of 30° C., the impurity increases. We prefer to control the reaction temperature by controlling the rate of epoxide addition; if too much epoxide is added too rapidly, then the reaction becomes exothermic and the temperature can exceed 30° C.

Accordingly, we prefer to begin to add epoxide at about 0° to 5° C., and add epoxide at a rate which maintains the exothermic reaction mixture below 25° C. After the epoxide is completely added, then we allow the temperature to raise to not more than 30° C., and then maintain the temperature for approximately four hours, or until the reaction is completed.

On completion of reaction, the reaction mixture is cooled to 0° to 5° C. Cooling the reaction mass before the following step (quenching the mass with water) is important because quenching the mass with water is exothermic and so, to maintain a maximum temperature under about 25 ° C., the reaction mass must be cooled to less than this.

The reaction mass is quenched by 2.25 volume of water, maintaining the temperature at not more than 25 ° C. The product is then extracted using 3 volumes of toluene. The toluene layer is washed with water for removal of isopropyl amine; after three water washes, isopropyl amine content is less than 0.5%. Traces of isopropyl amine are removed by maintaining under vacuum, below 25° C. It is necessary to eliminate traces of isopropyl amine at a temperature below 25° C., as the presence of isopropyl amine during distillation of toluene above 25° C. leads to the formation of an impurity at RRT 1.54 (as measured by gas chromatography). By following the process described above, this impurity formation is avoided.

In case when isopropyl amine remains in the traces, the impurity is detected at RRT 1.54 (as measured by gas chromatography).

Thus, following process as per present invention, formation of the impurities at RRT 0.35 & RRT 1.54 are under control, which leads to yield highly pure Metoprolol base in higher yields.

The analysis of a sample of the toluene layer confirms the absence of isopropyl amine in the toluene. The toluene is distilled out at temperature 30° to 40° C. under vacuum.

The residue of metoprolol base so obtained shows a purity of greater than 99%, and a yield of which is in range of 88 to 89% of the theoretical stoicheometric maximum yield.

Step 3A: Metoprolol Succinate Salts from Metoprolol Base

The metoprolol base is dissolved in seven volumes of acetone. Once dissolved, carbon treatment is done at 45° C. A solution of succinic acid in stoichiometric proportion to the Metoprolol base (i.e., in a 1:2 ratio) is prepared in twenty volumes of acetone; succinic acid solution is added to metoprolol base solution and adjusted to pH 7.2±0.1. This mixture is then refluxed for 4-5 hours at about 30° C. After refluxing, the mixture is then cooled to 26° C. This same temperature (26° C.) of the reaction mixture is then maintained with stirring for two hours, and is then filtered. The metoprolol succinate salt obtained is purified by crystallization from three volumes of methanol.

The resultant Metoprolol succinate is obtained in a yield of 72 to 75% of theoretical (Stoicheometric) maximum, with a purity of more than 99.8% (as shown by High Pressure Liquid Chromatography) and with no single impurity accounting for more than 0.1% of the total product.

Step 3B: Metoprolol Tartrate Salts from Metoprolol Base

The metoprolol base from the above-described step 2 is dissolved in seven volumes of acetone and added activated charcoal, heated to 45° C., stirred for 30 minutes, and then filtered with charcoal.

A solution of tartaric acid in acetone is prepared by dissolving tartaric acid in stoichiometric proportion (i.e., a ratio of 1:2 to the Metoprolol base) to the metoprolol base in 18 volumes of acetone, by refluxing. The tartaric acid solution is added to the Metoprolol base solution under refluxing conditions, and the acidity is adjusted to a pH of 6.2±0.1.

This reaction mixture is refluxed for 4 hours and cooled to 26° C. The reaction mixture is stirred at 26° C. for 2 hours and filtered. The yield of Metoprolol tartrate obtained is 83 to 83% of the theoretical (Stoicheometric) maximum yield.

The Metoprolol tartarate is then crystalized from nine volumes of isopropyl alcohol. The pure Metoprolol tartarate is obtained in a yield of 72 to 73% of the theoretical (stoicheometric) maximum yield, with a purity greater than 99.8% as measured by High Pressure Liquid Chromatography, with no other single impurity measuring more than 0.1% of the total product.

While we disclose here specific examples of our invention, it is possible for one of skill in the art to use our teachings to develop variations on our specific examples to achieve the goals of our invention. Thus, we intend the scope of our patent to be defined not by the specific examples here recited, but by the claims we append here and their legal equivalents.

1. A process for obtaining an aryloxypropanolamine of the chemical name 1-[4-)2-methoxyethyl)-phenoxy]-3-[(1-methylethyl)amino]-2-propanol of the formula 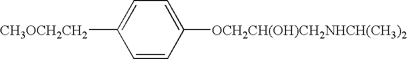
comprising:
A) combining 4-(2-methoxyethyl)phenol with epichlorhydrin;
B) reacting said combination of 4-(2-methoxyethyl)phenol and epichlorhydrin in an alkaline aqueous medium;
C) extracting and washing the organic phase reaction product of Step B with water at pH 7.5±0.5; and
D) obtaining a crude reaction product comprising 3-[4-(2-methoxyethyl)phenoxy]-1,2-epoxypropane;
E) combining said 3-[4-(2-methoxyethyl)phenoxy]-1,2-epoxypropane with isopropanolamine;
F) reacting said combination of 3-[4-(2-methoxyethyl)phenoxy]-1,2-epoxypropane and isopropanolamine in an aqueous medium at a temperature about 30° C., to obtain 1-[4-)2-methoxyethyl)-phenoxy]-3-[(1-methylethyl)amino]-2-propanol.

2. The process of claim 1, wherein:
A) said 4-(2-methoxyethyl)phenol and said epichlorhydrin are combined in a molar ratio of about 1:1.31.

3. The process of claim 2, wherein:
B) said reacting 4-(2-methoxyethyl)phenol and epichlorhydrin is at 42.5±2.5° C.; and
D) said crude reaction product is composed of about 97 to 99% of 3-[4-(2-methoxyethyl)phenoxy]-1,2-epoxypropane.

4. The process of claim 3, wherein:
E) said 3-[4-(2-methoxyethyl)phenoxy]-1,2-epoxypropane and isopropanolamine are combined in a molar ratio of about 1:5.25.

5. The process of claim 4, further comprising:
G) extracting said 1-[4-)2-methoxyethyl)-phenoxy]-3-[(1-methylethyl)amino]-2-propanol from said aqueous reaction medium with a polar solvent at a temperature of not more than about 25° C.; and
H) removing said solvent by distillation under reduced pressure.

6. The process of claim 5, further comprising:
I) combining said 1-[4-)2-methoxyethyl)-phenoxy]-3-[(1-methylethyl)amino]-2-propanol with succinic acid in a molar ratio of approximately 1:2 in a solution of pH about 7.2, and
J) isolating from said solution the succinate form of said 1-[4-)2-methoxyethyl)-phenoxy]-3-[(1-methylethyl)amino]-2-propanol.

7. The process of claim 5, further comprising:
I) combining said 1-[4-)2-methoxyethyl)-phenoxy]-3-[(1-methylethyl)amino]-2-propanol with tartaric acid in a molar ratio of approximately 1:2 in a solution of pH about 6.2; and
J) isolating from said solution the tartarate form of said 1-[4-)2-methoxyethyl)-phenoxy]-3-[(1-methylethyl)amino]-2-propanol.

8. A product of the chemical name 1-[4-)2-methoxyethyl)-phenoxy]-3-[(1-methylethyl)amino]-2-propanol of the formula 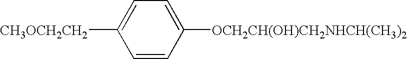
made by a process comprising:
A) combining 4-(2-methoxyethyl)phenol with epichlorhydrin;
B) reacting said combination of 4-(2-methoxyethyl)phenol and epichlorhydrin in an alkaline aqueous medium;
C) extracting and washing the organic phase reaction product of Step B with water at pH 7.5±0.5; and
D) obtaining a crude reaction product comprising 3-[4-(2-methoxyethyl)phenoxy]-1,2-epoxypropane;
E) combining said 3-[4-(2-methoxyethyl)phenoxy]-1,2-epoxypropane with isopropanolamine;
F) reacting said combination of 3-[4-(2-methoxyethyl)phenoxy]-1,2-epoxypropane and isopropanolamine in an aqueous medium at a temperature about 30° C., to obtain 1-[4-)2-methoxyethyl)-phenoxy]-3-[(1-methylethyl)amino]-2-propanol.

9. The product of claim 8, wherein:
A) said 4-(2-methoxyethyl)phenol and said epichlorhydrin are combined in a molar ratio of about 1:1.31.

10. The product of claim 9, wherein:
B) said reacting 4-(2-methoxyethyl)phenol and epichlorhydrin is at 42.5±2.5° C.; and
D) said crude reaction product is composed of about 97 to 99% of 3-[4-(2-methoxyethyl)phenoxy]-1,2-epoxypropane.

11. The product of claim 10, wherein:
E) said 3-[4-(2-methoxyethyl)phenoxy]-1,2-epoxypropane and isopropanolamine are combined in a molar ratio of about 1:5.25.

12. The process of claim 11, further comprising:
G) extracting said 1-[4-)2-methoxyethyl)-phenoxy]-3-[(1-methylethyl)amino]-2-propanol from said aqueous reaction medium with a polar solvent at a temperature of not more than about 25° C.; and
H) removing said solvent by distillation under reduced pressure.

13. The process of claim 12, further comprising:
I) combining said 1-[4-)2-methoxyethyl)-phenoxy]-3-[(1-methylethyl)amino]-2-propanol with succinic acid in a molar ratio of approximately 1:2 in a solution of pH about 7.2, and
J) isolating from said solution the succinate form of said 1-[4-)2-methoxyethyl)-phenoxy]-3-[(1-methylethyl)amino]-2-propanol.

14. The process of claim 12, further comprising:
I) combining said 1-[4-)2-methoxyethyl)-phenoxy]-3-[(1-methylethyl)amino]-2-propanol with tartaric acid in a molar ratio of approximately 1:2 in a solution of pH about 6.2; and
J) isolating from said solution the tartarate form of said 1-[4-)2-methoxyethyl)-phenoxy]-3-[(1-methylethyl)amino]-2-propanol.

15.
